(19) United States
(12) Patent Application Publication
(10) Pub. No.: US 2003/0232064 A1
(43) Pub. Date: Dec. 18, 2003

(54) Suppression of human activity in an enclosed space

(76) Inventors: William H. Burns JR., Orchard Park, NY (US); John C. McNeirney, Fairburn, GA (US)

Correspondence Address: Martin G. Linihan, Hodgson Russ LLP, One M&T Plaza, Suite 2000, Buffalo, NY, 14203-2391, US

(21) Appl. No.: 10/272,922

(22) Filed: Oct. 17, 2002

Related U.S. Application Data
(60) Provisional application No. 60/330,061, filed on Oct. 17, 2001.

(51) Int. Cl. A61K 31/08; A01N 25/00

(52) U.S. Cl. 424/405; 514/722

(57) ABSTRACT

The present invention provides a method for suppressing human activity in enclosed spaces. More particularly, the invention provides a method for the introduction of anesthesia into an enclosed space for the purpose of rendering occupants of the enclosed space unconscious.

### CROSS REFERENCE TO A RELATED APPLICATION

Applicants hereby claim priority based on U.S. Provisional Patent Application No. 60/330,061, filed Oct. 17, 2001 entitled “Suppression of Human Activity in an Enclosed Space” which is incorporated herein by reference.

### FIELD OF THE INVENTION

This invention relates to the field of anesthetics and the suppression of human activity in enclosed spaces. More particularly, this invention provides method and system for the delivery of anesthetics for suppression of human activity in enclosed spaces.

### BACKGROUND OF THE INVENTION

In certain situations, it is advantageous to render certain human individuals in an enclosed space unconscious so that these individuals can be physically restrained. This invention describes a method for meeting this need using inhalation anesthetic agents introduced into the enclosed space.

In emergency situations, it is extremely difficult to selectively immobilize the desired individuals when they are within a group of other individuals. However, this invention makes it possible to render all individuals in the enclosed space unconscious, allow time for physical restraints to be applied to the desired individuals and allow the full recovery of consciousness in the all individuals with minimal side effects.

Inhalation anesthetic agents are commonly used in surgical procedures to render a patient unconscious and tolerant of pain. These same anesthetic agents can be used, under properly controlled conditions, to cause humans to lose consciousness (induction), limit the depth of anesthetic effect so that they do not suffer respiratory collapse, and be able to regain consciousness with minimal side effects once the anesthetic agent is reduced/removed from the ambient “air” (“air” in this instance means the gaseous mixture the human is breathing). These commercially available inhalation anesthetic agents have already been approved for use by FDA, thus their safety and efficacy are well established.

Inhalant anesthetic agents are attractive for this purpose since they are expelled from the body by exhaling the anesthetic agent and therefore do not require long recovery times.

An inhalation anesthetic agent for this use may have the following properties. The anesthetic should: be tasteless, odorless and colorless; not cause involuntary respiratory tract reactions such as laryngospasms or swelling of the airways into the lungs; have a MAC (the Minimum Alveolar Concentration, the volume based percent at which 50% of humans lose consciousness) of 2% of less (this property is directly related to the time, at MAC, required for the individual to lose consciousness. MAC is different for each anesthetic agent); be not sensed at high concentrations, 3 to 5 times MAC, due to changes in the density of “air” being inhaled; and, have a low blood gas solubility such that the normal release of Carbon Dioxide from the blood stream during respiration will tend to purge the anesthetic agent from the lungs.

### SUMMARY OF THE INVENTION

The present invention provides a method for suppressing human activity in enclosed spaces. More particularly, the invention provides a method and system for the introduction of anesthesia into an enclosed space for the purpose of rendering occupants of the enclosed space temporarily unconscious.

### BRIEF DESCRIPTION OF THE DRAWINGS

FIG. 1 is a block diagram showing an embodiment of the invention.

### DETAILED DESCRIPTION OF THE INVENTION

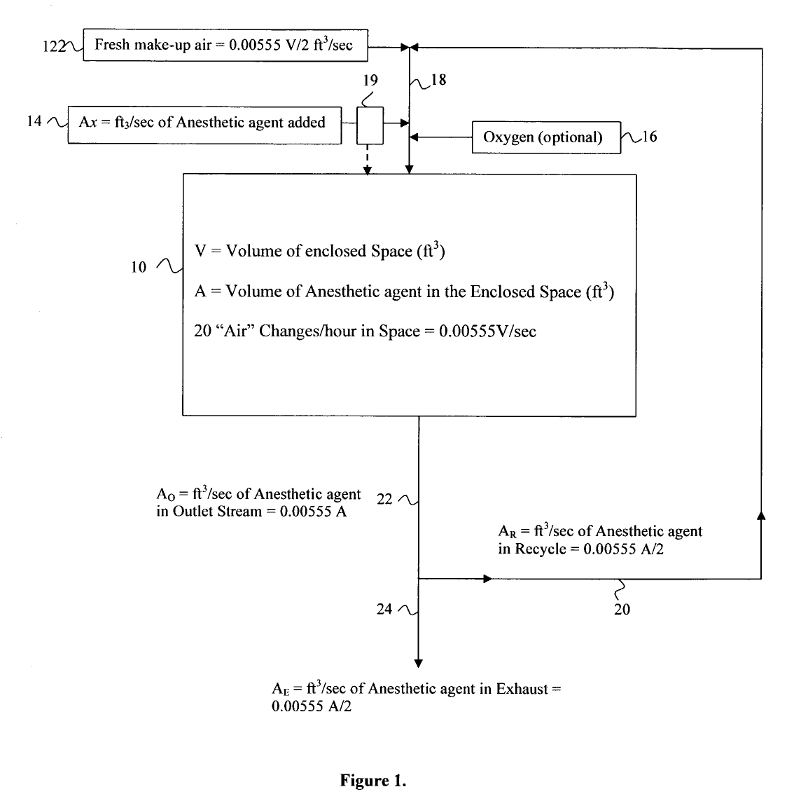

FIG. 1 shows the introduction of an anesthetic agent from a suitable source 14 into an enclosed space 10 either by direct release of the anesthetic agent into the space 10 as indicated by the dotted line flow path in FIG. 1, or by introduction of the anesthetic agent into an air exchange inlet stream 18 with fresh air 12. The introduction of the anesthetic is performed at a rate sufficient to cause the concentration of anesthetic agent in the enclosed space 10 to rapidly exceed 2 times MAC (this concentration is sufficient to induce 100% of the general population). The concentration needed for rapid induction (3 minutes or less) may be in the range of 4 to 6 times MAC. Both the rate and the duration of the flow are controlled by a suitable control means 19. The air from the enclosed space 10 may be shunted through an outlet stream 22 and then through a recycle route 20 or an exhaust 24. The enclosed space is occupied by individuals, typically humans, susceptible to the effect of anesthetic.

When the concentration of anesthetic agent reaches the desired multiple of MAC, the flow of the anesthetic agent may be reduced or shut off. The anesthetic agent concentration then drops, by the normal air exchange function, to levels such that it maintains the unconscious state but the concentration is such that it will not induce unconsciousness. At this time, other people can enter the space and apply appropriate physical restraints to the certain target individuals before they regain consciousness. Recovery times of individuals subjected to the anesthetic agent will be on the order of 15 to 25 minutes after the anesthetic agent concentration in the enclosed space falls below 0.3 times MAC.

### EXAMPLE I

Use in a Passenger Aircraft Cabin

While the anesthetic agent used in this example is Sevoflurane, the example is not meant to be limiting in any way. Sevoflurane is stable over a wide range of temperature. It can be stored in under pressure in cylinders for long periods (5-7 years) without measurable degradation.

Cylinders of the liquid anesthetic agent may be positioned in a secure area in the cargo hold of the airliner. Sevoflurane boils at 58.6° C. at 1 atmosphere and is stable at higher temperatures. The cylinders may be heated, such that the pressure generated by the liquid in the cylinder is sufficient to introduce the desired volume of vapor into the aircraft air handling system. Alternatively, pressurized liquid agent may be introduced into the cabin system using an atomization system, several of which are well known.

The enclosed space 10 of FIG. 1 is analogous to the passenger cabin in a commercial aircraft. The aircraft cockpit may be equipped with oxygen systems separate from the system that is available in the passenger cabin.

The pressurized anesthetic agent may be released into the passenger cabin under control of the aircraft commander (First Pilot) after the cockpit crew are on their oxygen supply.

The anesthetic agent may also be mixed with pure oxygen prior to introduction into the passenger cabin to avoid hypoxia during the period that the passengers are unconscious.

Control valves (or fixed orifice flow controls) in the anesthetic agent release system, i.e., in the control means 19, may be used to allow the desired amount of anesthetic agent to enter the cabin air make-up stream and continue to add anesthetic agent to the cabin air so that the desired concentration of anesthetic agent, sufficient to induce unconsciousness, is present in the cabin airspace. At the end of a timed introduction, based on the total volume of the passenger cabin, the anesthetic agent introduction system may reduce or shut off the introduction of the anesthetic agent into the cabin air system.

Since current aircraft ventilation systems have air entering at the cabin ceiling, exhausting at the cabin floor, and the anesthetic agent is heavier than air, the concentration of anesthetic agent will be higher at floor level. The result is that the passengers, who would be seated, would receive an average dose of anesthetic agent. Anyone that was standing when they lost consciousness, and fell to the floor, would receive a higher dosage of anesthetic agent which may be a desirable outcome because a hijacker is more likely to be standing.

The normal aircraft ventilation system might then reduce the concentration of anesthetic agent to a level such that the cockpit crew could enter the passenger cabin, without the need for either a gas mask or separate breathing apparatus, and physically restrain the desired individuals.

Based on the published properties and anesthetic performance of Sevoflurane, it is believed that the cockpit crew would have 15-20 minutes to apply physical restraints to the desired individuals.

In the same time period, it is believed the passengers would regain consciousness with minimal side effects. They would be slightly disoriented for several seconds but would, typically, fully recover without life threatening side effects.

While the invention has been illustrated for use in aircraft in the event of hijacking, the invention has potential use in other environments, including for example prisons, and wherein individuals, typically humans but possibly also animals, occupy and enclosed space into which the anesthetic can be introduced and wherein it is desired to render the occupants temporarily unconscious, typically for the purpose of restraining selected ones of the occupants.

Although embodiments of the invention have been described herein, the invention is not limited to such embodiments. The claims which follow are directed to the invention, and are intended to further describe the invention, but their literal language is not intended to limit the scope of the invention.

1. In combination:
a) an enclosed space surrounding a plurality of occupants wherein the occupants are susceptible to the effects of an anesthetic agent such that upon exposure to a sufficient amount of the anesthetic agent the occupants are rendered unconscious; and
b) a means for delivering an amount of anesthetic to the enclosed space sufficient to render the occupants unconscious.

2. The combination of claim 1 wherein the anesthetic is selected from the group consisting of halogenated methyl-methyl, methyl-ethyl, methyl-butyl, methyl-propyl, ethyl-ethyl, ethyl-butyl or ethyl-propyl ethers that are know to have anesthetic like properties.

3. The combination of claim 1, wherein the anesthetic agent is selected from the group consisting of: Sevoflurane, Desflurane, Halothane, Isoflurane, Enflurane, either used alone or in combination.

4. The combination of claim 1, wherein a mixture of anesthetic agents is used.

5. The combination of claim 1, wherein oxygen is introduced into the enclosed space in addition to the anesthetic agent, or mixture of agents.

6. The combination of claim 1, wherein the rate of introduction of the anesthetic agent, or mixture of anesthetic agents, into the enclosed space is such that unconsciousness of all occupants is achieved in 3 minutes or less.

7. The combination of claim 1, wherein the means for delivering an amount of anesthetic includes means for controlling the rate and duration of flow of anesthetic.

8. A method of suppressing activity in an enclosed space comprising:
a) providing an enclosed space to surround a plurality of occupants wherein the occupants are susceptible to the effects of an anesthetic agent such that upon exposure to a sufficient amount of the anesthetic agent the occupants are rendered unconscious; and
b) delivering an amount of anesthetic to the enclosed space sufficient to render the occupants unconscious.

9. The method of claim 8 wherein the anesthetic is selected from the group consisting of halogenated methyl-methyl, methyl-ethyl, methyl-butyl, methyl-propyl, ethyl-ethyl, ethyl-butyl or ethyl-propyl ethers that are know to have anesthetic like properties.

10. The method of claim 8, wherein the anesthetic agent is selected from the group consisting of: Sevoflurane, Desflurane, Halothane, Isoflurane, Enflurane, either used alone or in combination.

11. The method of claim 8, wherein a mixture of anesthetic agents is used.

12. The method of claim 8, wherein oxygen is introduced into the enclosed space in addition to the anesthetic agent, or mixture of agents.

13. The method of claim 8, wherein the rate of introduction of the anesthetic agent, or mixture of anesthetic agents, into the enclosed space is such that unconsciousness of all occupants is achieved in 3 minutes or less.

14. The method of claim 8, wherein the means for delivering an amount of anesthetic includes means for controlling the rate and duration of flow of anesthetic.
